(19) United States
(12) Patent Application Publication
(10) Pub. No.: US 2024/0076311 A1
(43) Pub. Date: Mar. 7, 2024

(54) METHOD FOR SYNTHESIZING PEPTIDE THIOESTERS AND HEAD-TO-TAIL AMIDE CYCLIC PEPTIDE THEREOF

(71) Applicant: Jiangsu Genscript Biotech Co., Ltd., Zhenjiang (CN)

(72) Inventors: Zhili Chen, Zhenjiang (CN); Tongqiang Tao, Zhenjiang (CN); Baofeng Tan, Zhenjiang (CN); Xin Zhang, Zhenjiang (CN)

(73) Assignee: Jiangsu Genscript Biotech Co., Ltd., Zhenjiang, JS (CN)

(21) Appl. No.: 18/259,476

(22) PCT Filed: Dec. 27, 2021
(86) PCT No.: PCT/CN2021/141673

§ 371 Date: Jun. 27, 2023

(30) Foreign Application Priority Data
Dec. 28, 2020 (CN) 202011579916.9

(51) Int. Cl. C07K 1/113; C07K 1/04; C07K 1/06; C07K 1/107

(52) U.S. Cl. CPC C07K 1/113; C07K 1/04; C07K 1/061; C07K 1/1077

(57) ABSTRACT

A method for synthesizing peptide thioesters and a head-to-tail amide cyclic peptide thereof, which belongs to the technical field of chemical pharmaceuticals and fine chemical preparation. The method comprises the following steps: (1) using resin A as a carrier and using a solid-phase synthesis strategy to obtain a resin peptide; (2) cutting the resin to obtain a fully protected peptide; (3) performing an esterification reaction with p-chlorophenyl thiophenol in a TCFH/alkali condensation system to generate p-chlorophenyl thioester; (4) removing the protecting group to obtain a peptide thioester; and (5) further cyclizing to obtain a head-to-tail cyclic peptide. The preparation of the thioester peptide and the head-to-tail amide cyclic peptide provides a simple technical route with wide universality and a high yield, and has a wide range of applications in the technology of chemical pharmaceuticals and fine chemical preparation.

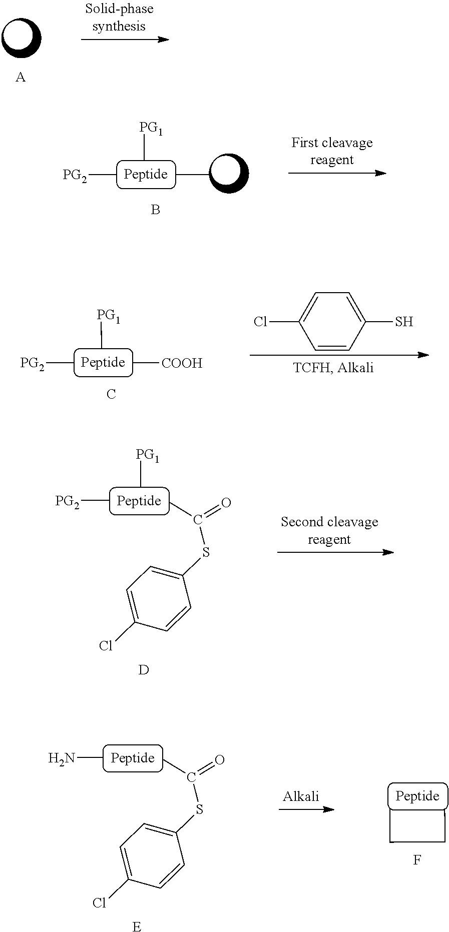

### CROSS-REFERENCE TO RELATED APPLICATIONS

This application claims priority to Chinese Patent Application No. 202011579916.9, filed with the China National Intellectual Property Administration on Dec. 28, 2020, which is incorporated herein by reference in its entirety.

### TECHNICAL FIELD

The present invention relates to the technical fields of chemical pharmaceutical and fine chemical preparation, and particularly relates to methods for synthesizing a peptide thioester and a head-to-tail amide cyclic peptide thereof.

### BACKGROUND ART

Natural cyclic peptides play an important role in pharmacological studies and have a strong resistance to proteases and a reduced conformational flexibility compared with straight-chain peptides. The natural cyclic peptides have met the criteria of stability, efficacy, and selectivity of drugs, for example, many natural cyclic peptides, such as, vancomycin, cyclosporin A, and romidepsin, have been developed into drugs. However, some straight-chain peptides have a large steric hindrance and a low activity, and are not easy to be cyclized. However, a peptide thioester is a highly active compound, and can be used to obtain a cyclic peptide with a high yield, and a head-to-tail cyclic peptide difficult to be cyclized may be formed therefrom.

In 1994, Kent group directly synthesized a polypeptide with the C-terminal of thiocarboxylic acid by means of a solid-phase method and subsequently converted the polypeptide into a thioester by means of benzyl bromide or an Ellman's reagent. However, the reaction steps are more and the reagents are relatively expensive; thus, the method is not suitable for mass production (Dawson P E, Muir T W, Kent S B. Synthesis of proteins by native chemical ligation. Science, 1994, 266. 776-779).

In 2001, Hilvert group developed a solid-phase synthesis method for a peptide thioester, wherein carboxypropyl sulfonamide is used as a linking molecule, after a polypeptide synthesis was chemically completed by means of Fmoc, the sulfonamide was alkylated with iodeacetonitrile or trimethylsilyl azomethane (TMS₂CHN₂), then cleaved from a resin with a thiol and subjected to an thioesterification with LiBr/THF, and a side chain protecting group was removed with TFA to obtain an unprotected polypeptide thioester. However, the synthesis of peptide thioester had many steps, the operation was complicated, the reaction conditions were harsh, and the reaction reagents were not easy to obtain and expensive, and the yield of peptide thioester is low (Quaderer R and Hilvert D. Improved Synthesis of C-Terminal Peptide Thioesters on “Safety-Catch” Resins Using LiBr/THF. Org Lett. 2001, 3, 3181-3184).

In 2009, Houghten group used a “volatile” thioester silica gel as a solid-phase carrier to synthesize straight-chain peptides in sequence, obtained a peptide thioester under HF cleavage, and finally obtained a head-to-tail cyclized product with a high yield in a mixture of acetonitrile and a 1.5 M imidazole aqueous solution. However, the thioester silica gel carrier was not commonly used and the strongly corrosive HF was also used (Li Y M, Yongye A and Houghten R A. Synthesis of Cyclic Peptides through Direct Aminolysis of Peptide Thioesters Catalyzed by imidazole in Aqueous Organic Solutions. J.Comb. Chem. 2009, 11, 1066-1072).

in 2014, Eberle group coupled p-chlorothiophenol with a fully protected polypeptide with the C-terminal of carboxylic acid under the catalysis of PyBop to obtain a peptide thioester, followed by removing a protecting group therefrom, and synthesizing a head-to-tail ring thereof under the catalysis of an alkali. However, the synthesis of a peptide thioester by means of PyBop has a relatively low yield, and the peptide thioester is not easy to purify (Agrigento P, Albericio F and Eberle M. Facile and Mild Synthesis of Linear and Cyclic Peptides via Thioesters. Org. Lett. 2014, 16, 3922-3925).

Therefore, simple and efficient methods for synthesizing a peptide thioester and a head-to-tail amide cyclic peptide thereof still need to be developed.

### SUMMARY OF THE INVENTION

In view of the disadvantages in the prior art, an object of the present invention is to provide methods for synthesizing a peptide thioester and a head-to-tail amide cyclic peptide thereof. Under the catalysis of a cheap and easily available coupling reagent of TCFH, p-chlorothiophenol and N-terminal Boc-protected fully protected peptide are subjected to an esterification reaction to obtain a series of high-yield and high-purity peptide thioesters, and finally, under the action of an alkali, a head-to-tail amide cyclic peptide is obtained.

The present invention provides a simple and convenient method for synthesizing a peptide thioester and a head-to-tail amide cyclic peptide thereof: A synthetic route is as follows:

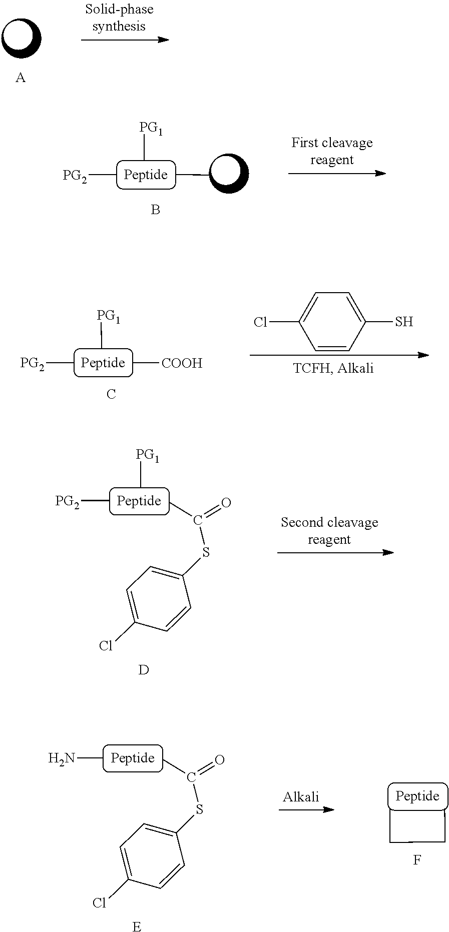

wherein the peptide is a peptide chain and PG₁ is all the protecting groups on a

side chain of the peptide chain; and PG₂, is a protecting group at the N-terminal of the peptide chain.

The preparation methods of the present invention are further specifically described below. However, it should he understood that the present invention is not limited to the specific reaction conditions (e.g., a solvent, an amount of a compound used, a reaction temperature, a time required by a reaction, etc.) given below.

A first technical problem to be solved by the present invention is: to provide a simple and convenient method for synthesizing a peptide thioester.

To solve the first technical problem, the technical solution used in the present invention is: a simple and convenient method for synthesizing a peptide thioester. The method comprises the following steps:
(1) preparation of a resin peptide B: using a resin A as a carrier and sequentially coupling corresponding amino acids in an order from the C-terminal to the N-terminal according to a target sequence by means of solid-phase synthesis to obtain a resin peptide B;
(2) preparation of a fully protected peptide C: cleaving the resin from the resin peptide B obtained in step (1) with a first cleavage reagent to obtain a fully protected peptide C;
(3) preparation of a fully protected peptide thioester D: subjecting the fully protected peptide C obtained in step (2) to an esterification reaction with p-chlorothiophenol under the action of a coupling agent of TCFH and an alkali in a solvent to obtain a fully protected peptide thioester D; and
(4) preparation of a peptide thioester E: under the action of a second cleavage reagent, removing a protecting group from the fully protected peptide thioester D obtained in step (3) to obtain a peptide thioester E,

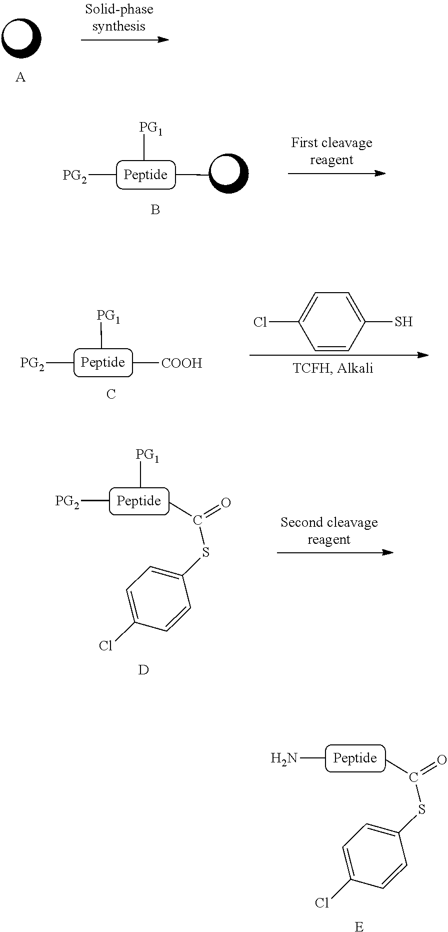

Wherein the peptide is a peptide chain and PG₁ is all the protecting groups on a side chain of the peptide chain; and is a protecting group at the N-terminal of the peptide chain.

In some examples of the present invention, in step (1), the PG₂ is Boc, the peptide chain is a straight chain, and a procedure for synthesizing the resin peptide B is that the last amino acid of the coupling is a Boc-protected amino acid or an N-terminal Boc protection of the peptide chain is performed by means of di-tert-butyl dicarbonate.

In some examples of the present invention, in step (2), the first cleavage reagent is a conventional cleavage reagent in the art, such as a TFA/DCM solution with a volume fraction of 1% and a mixed solution of trifluoroethanol, acetic acid, and DCM at a volume ratio of 1:2:7.

In some examples of the present invention, the first cleavage reagent is a dichloromethane solution of trifluoroisopropanol, a volume fraction of the trifluoroisopropanol in the dichloromethane is 10%-90%, and the cleavage is performed for 1-5 tunes with each cleavage time of 0.5 h-6 h. Preferably, the volume fraction of the trifluoroisopropanol in the dichloromethane is 33%; and the cleavage is performed for 3 times with each cleavage time of 1 h.

In some examples of the present invention, in step (3), the alkali is selected from at least one of DIPEA or NMI; and a molar ratio of the fully protected peptide C, the p-chlorothiophenol, the TCFH, and the alkali is 1:(1-2):(1-3):(2-5); and preferably, the alkali is NMI, and the molar ratio of the fully protected peptide C, the p-chlorothiophenol, the TCFH, and the alkali is 1:1.2:1.5:4.

In some examples of the present invention, in step (3), the solvent is one or more of DMF, DMSO, or DMA; a concentration of the fully protected peptide C in the solvent is 0.01 M-0.2 M; and preferably, the solvent is DMF, and preferably, the concentration of the fully protected peptide C in the solvent DMF is 0.01 M-0.2 M, more preferably, 0.1 M.

In some examples of the present invention, in step (3), the coupling reaction is performed for a time of 4 h-24 h, preferably, 16 h-24 h, more preferably, 16 h.

In some examples of the present invention, the coupling reaction is performed at a temperature of 20° C-100° C., preferably, 25° C-50° C., more preferably, 30° C.

In some examples of the present invention, in step (4), the second cleavage reagent is a mixed solution of one or more of EDT, phenol, thioanisole or H₂O with TFA; preferably, the second cleavage reagent is a mixed solution of the TFA, the EDT, the phenol, the thioanisole, and the H₂O; and a volume ratio of the TFA, the EDT, the phenol, the thioanisole, and the H₂O in the mixed solution is (50-95):(1-12.5):(1-12.5):(1-12.5):(1-12.5), preferably, 87.5:5:2.5:2.5:2.5.

A second technical problem to be solved by the present invention is: to provide a method for synthesizing a head-to-tail amide cyclic peptide.

To solve the second technical problem, the technical solution provided by the present invention is: a method for synthesizing a head-to-tail amide cyclic peptide.

The method comprises the following steps:
(1) preparing a peptide thioester E by the method of the present invention above; and
(2) under the action of an alkali, cyclizing the peptide thioester E prepared in step (1) in a solvent to obtain a head-to-tail amide cyclic peptide F,

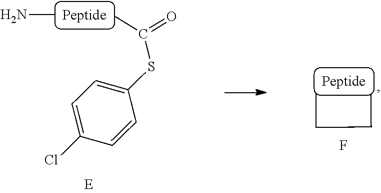

wherein the peptide is a peptide chain.

In some examples of the present invention, in step (2), the alkali is one or more of DIPEA, DBU, imidazole, or NMI; and a molar ratio of the peptide thioester E to the alkali is 1:(2-5).

In some examples of the present invention, in step (2), the solvent is one or more of DMF, DMSO, or DCM; and a concentration of the peptide thioester E in the solvent is 0.001 M-0.01 M.

Preferably, in step (2), the alkali is DIPEA; a molar ratio of the peptide thioester E to the alkali is 1:3; and the solvent is DMF, and a concentration of the peptide thioester E in the solvent is 0.0025 M.

In the present invention, unless otherwise specified, scientific and technical terms used herein have the meanings commonly understood by a person skilled in the art. Besides, the laboratory operating steps used herein are all conventional steps widely used in the corresponding field. Meanwhile, in order to better understand the present invention, the definitions and explanations of related terms are provided below.

The terms “cyclic peptide”, “head-to-tail amide cyclic peptide”, and “head-to-tail cyclic peptide” all refer to a polypeptide that is cyclic with an amide bond between the N-terminal and the C-terminal.

The term “solid-phase synthesis” refers to a synthesis method for linking reactants on an insoluble solid-phase carrier.

A nomenclature used to define a peptide is common in the art, wherein the N-terminal amino group appears on the left side and the C-terminal carboxyl group appears on the right side. Natural amino acids refer to one of natural amino acids found in a protein, namely Gly, Ala, Val, Len, Ile, Ser, Thr, Lys, Arg, Asp, Asn, Glu, Gln, Cys, Me, Phe, Tyr, Pro, Trp, and His.

The amino acids have isomer forms, Unless otherwise indicated, the amino acids are in an L form.

A hyphen or a suffix “-OH” and “—NH₂” after parentheses refer to a free acid and amide form of a polypeptide or an amino acid, respectively. For example: Fmoc-Val-OH refers to a free acid form of an N-terminal Fmoc-protected Val amino acid. NH₂-peptide refers to that the N-terminal amino acid at a peptide chain end is in an amide form.

In a chemical synthesis of a peptide, an appropriate protecting group is often used to protect reactive side chain groups of various amino acid moieties, which will prevent a chemical reaction from occurring at the position until finally the protecting group is removed.

The “fully protected peptide” in the present invention refers to a polypeptide with all active side chains protected by a protecting group. The “fully protected peptide thioester” in the present invention refers to a peptide thioester with all the active side chains protected by a protecting group. In addition, when the whole is reacted on a carboxyl group, it is also common to protect an a amino group of an amino acid or a fragment thereof, and then to cause a subsequent reaction at that position by selectively removing the a amino protecting group. Although specific protecting groups have been disclosed with regard to a solid-phase synthesis method, it should be noted that each amino acid may be protected by a protecting group commonly used for various amino acids in a solution-phase synthesis.

The solid-phase synthesis method begins at the C-terminal of a peptide by coupling a protected a-amino acid to a suitable resin. Such raw materials may be prepared as follows: An α-amino-protected amino acid is linked to a p-benzyloxybenzyl alcohol (Wang) resin or 2-Cl-Trt resin via an ester bond, or to a benihydrylamine (BHA) resin via an amide bond between Fmoc-Linker. The Fmoc solid-phase synthesis strategy is used in the present invention.

In a synthesis process of a polypeptide, a linking reaction of a first amino acid and a resin is different from a subsequent linking of an amino acid, particularly a substitution degree of the linked amino acid is directly related to a subsequent selection of a length of a polypeptide. A commercially available pre-loaded resin for a polypeptide synthesis, which is preloaded with a first amino acid, is used in the present invention. For example: A Fmoc-Ala-2-Cl-Trt resin and a Fmoc-Gly-2-Cl-Trt resin, etc. are purchased from GL Biochem company.

Beneficial effects are as follows:

the present invention provides an efficient and simple condensation system for synthesizing a peptide thioester.
The peptide thioester provided by the present invention is highly active, may form a head-to-tail amide cyclic peptide difficult to be cyclized, and has a wide applicability. The method for synthesizing a head-to-tail amide cyclic peptide of the present invention is simple to operate, and has a high yield of a head-to-tail amide cyclic peptide, a low cost, and a wide applicability.

### BRIEF DESCRIPTION OF THE DRAWINGS

The present invention will be more fully understood from the following detailed description in combination with the accompanying drawings:

FIG. 1 is an MS spectrum of compound F1;

FIG. 2 is an HPLC profile of compound F1;

FIG. 3 is an MS spectrum of compound F2;

FIG. 4 is an HPLC profile of compound F2;

FIG. 5 is an MS spectrum of compound F3; and

FIG. 6 is an HPLC profile of compound F3.

### DETAILED DESCRIPTION OF EMBODIMENTS

The technical solutions of the present invention are further illustrated in detail below by the examples in combination with. the accompanying drawings. However, the present invention is not limited to the following examples.

The raw materials or reagents used in the present invention are commercially available, unless specially specified. The common chemical reagents are shown in. Table 1:

TABLE 1
Common chemical reagents

| Raw materials | Chinese name | CAS | Factory |
|---|---|---|---|
| Fmoc-Ala-OH | N-fluorenylmethoxycarbonyl- | 35661-39-3 | GL |
| | L-alanine | | Biochem |
| Fmoc-Cys(Trt)-OH | Fmoc-S-triphenylmethyl-L- | 103213-32-7 | GL |
| | cysteine | | Biochem |
| Fmoc-Asp(tBu)-OH | Fmoc-L-aspartic acid beta- | 71989-14-5 | GL |
| | tert-butyl ester | | Biochem |
| Fmoc-Glu(tBu)-OH | Fmoc-O-tert-butyl-L- | 71989-18-9 | GL |
| | glutamic acid | | Biochem |
| Fmoc-Phe-OH | Fmoc-L-phenylalanine | 35661-40-6 | GL |
| | | | Biochem |
| Fmoc-Gly-OH | Fmoc-glycine | 29022-11-5 | GL |
| | | | Biochem |
| Fmoc-His(Trt)-OH | N-Fmoc-N″- | 109425-51-6 | GL |
| | triphenylmethyl-L-histidine | | Biochem |
| Fmoc-Ile-OH | Fmoc-L-isoleucine | 71989-23-6 | GL |
| | | | Biochem |
| Fmoc-Lys(Boc)-OH | N-alpha- | 71989-26-9 | GL |
| | fluorenylmethoxycarbonyl-N- | | Biochem |
| | epsilon-tert-butoxycarbonyl- | | |
| | L-lysine | | |
| Fmoc-Leu-OH | Fmoc-L-leucine | 35661-60-0 | GL |
| | | | Biochem |
| Fmoc-Met-OH | Fmoc-L-methionine | 71989-28-1 | GL |
| | | | Biochem |
| Fmoc-Asn(Trt)-OH | Fmoc-N-triphenylmethyl-L- | 132388-59-1 | GL |
| | asparagine | | Biochem |
| Fmoc-Pro-OH | Fmoc-L-proline | 71989-31-6 | GL |
| | | | Biochem |
| Fmoc-Gln(Trt)-OH | Fmoc-N-triphenylmethyl-L- | 132388-59-1 | GL |
| | asparagine | | Biochem |
| Fmoc-Arg(Pbf)-OH | Fmoc-Pbf-L-arginine | 154445-77-9 | GL |
| | | | Biochem |
| Fmoc-Ser(tBu)-OH | FMOC-O-tert-butyl-L-serine | 71989-33-8 | GL |
| | | | Biochem |
| Fmoc-Thr(tBu)-OH | Fmoc-O-tert-butyl-L- | 71989-35-0 | GL |
| | threonine | | Biochem |
| Fmoc-Val-OH | Fmoc-L-valine | 68858-20-8 | GL |
| | | | Biochem |
| Fmoc-Trp(Boc)-OH | Fmoc-L-tryptophan (Boc)- | 143824-78-6 | GL |
| | OH | | Biochem |
| Fmoc-Tyr(tBu)-OH | Fmoc-O-tert-butyl-L-tyrosine | 71989-38-3 | GL |
| | | | Biochem |
| FMOC-D-LEU-OH | Fmoc-D-leucine | 114360-54-2 | GL |
| | | | Biochem |
| FMOC-D-TRP(BOC)-OH | N-alpha- | 163619-04-3 | GL |
| | fluorenylmethoxycarbonyl-N- | | Biochem |
| | in-tert-butoxycarbonyl-D- | | |
| | tryptophan | | |
| (Boc)₂O | Di-tert-butyl dicarbonate | 24424-99-5 | Energy |
| | | | Chemical |
| DIC | N,N′-diisopropyl | 693-13-0 | Tianjin |
| | carbodiimide | | Heowns |
| HOBT | 1-hydroxybenzotriazole | 2592-95-2 | Suzhou |
| | | | Highfine |
| | | | Biotech |
| PyBOP | 1H-benzotriazol-1- | 128625-52-5 | Tianjin |
| | yloxytripyrrolidylhexafluorophosphate | | Heowns |
| HATU | 2-(7-azobenzotriazole)-N,N,N′,N′- | 148893-10-1 | Suzhou |
| | tetramethyluronium | | Highfine |
| | hexafluorophosphate | | Biotech |
| TBTU | 2-(1H-benzotriazol-1-yl)- | 125700-67-6 | Suzhou |
| | 1,1,3,3-tetramethyluronium | | Highfine |
| | tetrafluoroborate | | Biotech |
| TCFH | N,N,N′,N′- | 94790-35-9 | Suzhou |
| | tetramethylchloroformamidine | | Highfine |
| | hexafluorophosphate | | Biotech |
| NMI | N-methylimidazole | 616-47-7 | Energy |
| | | | Chemical |
| DBU | 1,8-diazabicycloundec-7-ene | 6674-22-2 | Energy |
| | | | Chemical |
| 4-chlorothiophenol | p-chlorothiophenol | 106-54-7 | Energy |
| | | | Chemical |
| DIPEA | N,N-diisopropyl ethylamine | 7087-68-5 | Suzhou |
| | | | Highfine |
| | | | Biotech |
| DMF | N,N-dimethylformamide | 68-12-2 | Sinopharm |
| EDT | 1,2-ethanedithiol | 540-63-6 | Energy |
| | | | Chemical |
| TFA | Trifluoroacetic acid | 76-05-1 | Shanghai |
| | | | Aladdin |
| TFE | Trifluoroethanol | | |
| Phenol | Phenol | 108-95-2 | Shanghai |
| | | | Aladdin |
| Thioanisole | Thioanisole | 100-68-5 | Shanghai |
| | | | Aladdin |
| Fmoc-Ala-2-Cl-Trt resin | Fmoc-Ala-2-Cl-Trt resin | None | GL |
| | | | Biochem |
| Fmoc-Gly-2-Cl-Trt resin | Fmoc-Gly-2-Cl-Trt resin | None | GL |
| | | | Biochem |

Abbreviations used in the present invention have conventional meanings in the art. One-letter and three-letter abbreviations for various common amino acids are as suggested in PureAppl. Chem. 31, 639-645 (1972) and 40, 277-290 (1974), and conform to 37 CFR-1.822 C55FR 18245, May 1, 1990 and PCT rules (WIPÜ Standard ST. 23: Recommendation for the Presentation of Nucleotide and Amino Acid Sequences in Patent Applications and in Published Patent Documents). Common abbreviations and meanings thereof in present invention are shown in Table 2:

TABLE 2
Common abbreviations and meanings thereof in present invention

| | |
|---|---|
| Fmoc | 9-fluorenylmethoxycarbonyl |
| Boc | Tert-butoxycarbonyl |
| THF | Tetrahydrofuran |
| PyBop | (Benzotriazol-1-yloxy)tripyrrolidylphosphonium |
| | hexafluorophosphate |
| HMPA | Hexamethylphosphoramide |
| DMSO | Dimethyl sulfoxide |
| DMA | Dimethylacetamide |
| Pip | Piperidine |
| A (Ala) | Alanine |
| R (Arg) | Arginine |
| D (Asp) | Aspartic acid |
| C (Cys) | Cysteine |
| I (Ile) | Isoleucine |
| M (Met) | Methionine |
| L (Leu) | Leucine |
| W (Trp) | Tryptophan |
| G (Gly) | Glycine |

Unless otherwise stated, a feeding equivalent in the examples of the present invention is a molar equivalent, expressed in eq. for example, 1 eq represents one molar equivalent. The concentration unit M of the present invention is mol/L.

Example 1: Synthesis and purification of head-to-tail amide cyclic peptide F1 (AIMAA)

The example is directed to the synthesis and purification of a head-to-tail amide cyclic peptide AIMAA. The amino acids are subjected to a solid-phase synthesis by using Fmoc to protect an α-amino group. Specific synthesis steps are as follows:

(1) Preparation of a resin peptide B1: 1 g of a Fmoc-Ala-2-Cl-Trt resin is weighed and subjected to an operation A, i.e.: 20% of Pip/DMF is added to remove a Fmoc protecting group from the N-terminal, the mixture is reacted at 25° C. for 20 min, after the reaction, the reaction solution is washed with DMF for 5 times and detected by means of a ninhydrin detection reagent. If the detection is positive, the reaction is complete. Then the reaction solution is subjected to an operation B, i.e.: a mixed solution containing 1.5 mmol of DIC, 1.5 mmol of HOBT, and 1.5 mmol of an amino acid with a protecting group is added until a concentration is 0.5 M, the mixture is reacted at 25° C., after 1 hour, the reaction is finished. If the detection by means of the ninhydrin is negative, the reaction is complete. Then the reaction solution is washed 3 times with industrial DMF. Then the operations A and B are performed alternately, and only the corresponding amino acid is added in the operation B along with a synthesis sequence. Until the linking is to Ala, the operation A is performed, finally 1.5 mmol of (Boc)₂O and 1.5 mmol of DIPEA in dichloromethane are added, and the mixture is reacted for 30 min to obtain a resin peptide B1 as shown in the following figure.

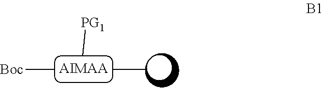

(2) Preparation of a fully protected peptide C1: a solution of trifluoroisopropanol in dichloromethane with a volume fraction of 33% as a first cleavage reagent is added into the resin peptide B1 obtained in step (1) for cleavage for 3 times with 1 h for each time, and the obtained product is freeze-dried to obtain a white powder, a fully protected peptide C1, with a theoretical molecular weight of 575.60 and an actually detected molecular weight of 575.21.

(3) Preparation of a fully protected peptide thioester D1:

100 mg of the fully protected peptide C1 obtained in step (2) is taken, 1.5 eq of TCFH and 4 eq of NMI in DMF (0.1 M) are respectively added, finally 1.2 eq of p-chlorothiophenol is added, the mixture is reacted at 30° C. for 16 h, the reaction solution is detected by means of HPLC, a yield is 82%, and an optimized experiment of the fully protected peptide thioester D1 is shown in Table 3.

(4) Preparation of a peptide thioester E1:

5 mL of a cleavage reagent (a mixed solution of TFA, EDT, phenol, thioanisole, and H₂O at a volume ratio of 87.5:5:2.5:2.5:2.5) is added into the fully protected peptide thioester D1 obtained in step (3), the mixture is reacted for 2 h, the reaction solution is settled with diethyl ether to obtain a crude product peptide thioester, and the peptide thioester is freeze-dried to obtain a powder, a peptide thioester E1, of 80 mg in total with a yield of 95%,

(5) Preparation of a head-to-tail amide cyclic peptide F1: 50 mg of the peptide thioester E1 obtained in step (4) is taken, 3.0 eq of DIPEA in DMF (0.0025 M) is added, the mixture is reacted for 12 h, and a reaction condition optimized experiment is shown in Table 4.

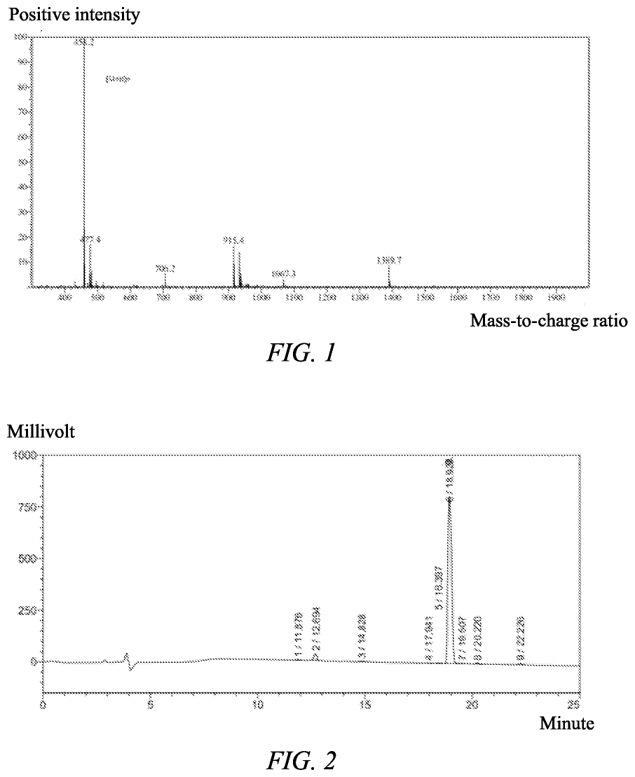

After a solvent is concentrated, the obtained product is settled with diethyl ether to obtain a crude product head-to-tail amide cyclic peptide, and the head-to-tail amide cyclic peptide is dissolved with a water/acetonitrile mixed solution, loaded into a high performance liquid chromatography for separation and purification with a mobile phase of H₂O/0.1TFA% and ACN/0.1.% TFA, and separated and purified with a gradient elution chromatography system via a C18 preparative column. A target fraction is collected. The purity of a collected target peak is detected by an analytical high performance liquid chromatography. A qualified sample is freeze-dried by means of liquid nitrogen, and finally the freeze-dried sample is freeze-dried in a vacuum freeze-drying machine to obtain 37 mg of a head-to-tail amide cyclic peptide compound F1 with a yield of 82% and a purity of 95%. The head-to-tail amide cyclic peptide compound F1 has a theoretical molecular weight of 457.59 and an actually detected molecular weight of 457.2. A detection is performed at 220 nm, using a C18 (4.6*250 mm) 5 μm chromatographic column, and with a linear gradient from 5% to 65%, acetonitrile (0.05% TEA) in water (0.065% TFA) at speed of 1 mL/min within 25 min, and t_{R}=18.9 min. An MS spectrum and an HPLC profile are as shown in FIG. 1 and FIG. 2.

Example 2 Synthesis and purification of head-to-tail amide cyclic peptide F2 (LWLLG)

The example is directed to the synthesis and purification of a head-to-tail amide cyclic peptide LWLLG. The amino acids are subjected to a solid-phase synthesis by using Fmoc to protect an α-amino group. Specific synthesis steps are as follows:

(1) Preparation of a resin peptide B2:

1 g of a Fmoc-Gly-2-C1-Trt resin is weighed and subjected to an operation A, i.e.: 20% of Pip/DMF is added to remove a Fmoc protecting group from the N-terminal, the mixture is reacted at 25° C. for 20 min, after the reaction, the reaction solution is washed with DMF for 5 times and detected by means of a ninhydrin detection reagent. If the detection is positive, the reaction is complete. Then the reaction solution is subjected to an operation B, i.e.: a mixed solution containing 1.5 mmol of DIC, 1.5 mmol of HOBT, and 1.5 mmol of an amino acid with a protecting group is added until a concentration is 0.5 M, the mixture is reacted at 2.5° C., after 1 hour, the reaction is finished. If the detection by means of the ninhydrin is negative, the reaction is complete. Then the reaction solution is washed 3 times with industrial DMF. Then the operations A and B are performed alternately, and only the corresponding amino acid is added in the operation B along with a synthesis sequence. Until the linking is to Ala, the operation A is performed, the final amino acid is Boc-Leu-OH, and the operation A is performed to obtain a resin peptide B2 as shown in the following figure.

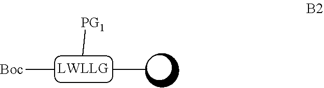

(2) Preparation of a fully protected peptide C2:

a solution of trifluoroisopropanol in dichloromethane with a volume fraction of 33% as a first cleavage reagent is added into the resin peptide B2 obtained in step (1) for cleavage for 3 times with 1 h for each time, and the obtained product is freeze-dried to obtain a white powder, a fully protected peptide C2, with a theoretical molecular weight of 800.75 and an actually detected molecular weight of 800.4.

(3) Preparation of a fully protected peptide thioester D2:

100 mg of the fully protected peptide C2 obtained in step (2) is taken, 1.5 eq of TCFH and 4 eq of NMI in DMF (0.1 M) are respectively added, finally 1.2 eq of p-chlorothiophenol is added, the mixture is reacted at 30° C. for 16 h, and the reaction solution is detected by means of HPLC, a yield is 86%.

(4) Preparation of a peptide thioester E2:

5 mL of a cleavage reagent (a mixed solution of TFA, EDT, phenol, thioanisole, and H₂O at a volume ratio of 87.5:5:2.5:2.5:2.5) is added into the fully protected peptide thioester D2 obtained in step (3), the mixture is reacted for 2 h, the reaction solution is settled with diethyl ether to obtain a crude product peptide thioester, and the peptide thioester is freeze-dried to obtain a powder, a peptide thioester E2: 85 mg with a yield of 94%.

(5) Preparation of a head-to-tail amide cyclic peptide F2:

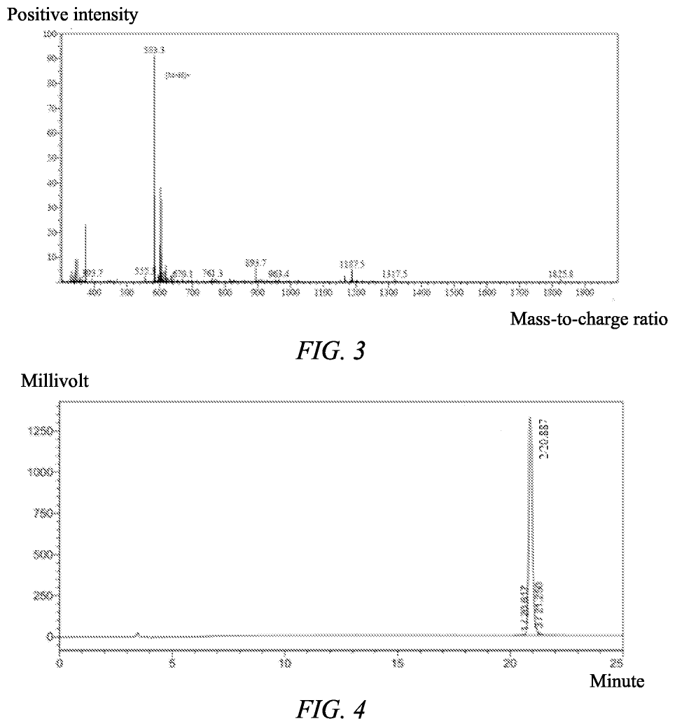

50 mg of the peptide thioester E2 obtained in step (4) is taken, 3.0 eq of DIPEA in DMF (0.0025 M) is added, the mixture is reacted for 12 h; after a solvent is concentrated, the obtained product is settled with diethyl ether to obtain a crude product head-to-tail amide cyclic peptide, and the head-to-tail amide cyclic peptide is dissolved with a water/acetonitrile mixed solution, loaded into a high performance liquid chromatography for separation and purification with a mobile phase of H₂O/0.1 TFA% and ACN/0.1% TFA, and separated and purified with a gradient elution chromatography system via a C18 preparative column. A target fraction is collected. The purity of a collected target peak is detected by an analytical high performance liquid chromatography. A qualified sample is freeze-dried by means of liquid nitrogen, and finally the freeze-dried sample is freeze-dried in a vacuum freeze-drying machine to obtain 35 mg of a head-to-tail amide cyclic peptide compound F2 with a yield of 78% and a purity of 98.9%. The head-to-tail amide cyclic peptide compound F2 has a theoretical molecular weight of 582.74 and an actually detected molecular weight of 582.3. A detection is performed at 220 nm, using a C18 (4.6*250 mm) 5 μm chromatographic column, and with a linear gradient from 5% to 65%, acetonitrile (0.05% TFA) in water (0.065% TFA) at speed of 1 mL/min within 25 min, and t_{R}=20.89 min An MS spectrum and an HPLC profile are as shown in FIG. 3 and FIG. 4.

Example 3 Synthesis and purification of head-to-tail amide cyclic peptide F3

The example is directed to the synthesis and purification of a head-to-tail amide cyclic peptide Gly{d-Leu} {d-Trp} {d-Leu} {d-Leu}. The amino acids are subjected to a solid-phase synthesis by using Fmoc to protect an α-amino group. Specific synthesis steps are as follows:

(1) Preparation of a resin peptide B3:

1 g of a Fmoc-Gly-2-Cl-Trt resin is weighed and subjected to an operation A, i.e.: 20% of Pip/DMF is added to remove a Fmoc protecting group from the N-terminal, the mixture is reacted at 25° C. for 20 min, after the reaction, the reaction solution is washed with DMF for 5 times and detected by means of a ninhydrin detection reagent. If the detection is positive, the reaction is complete. Then the reaction solution is subjected to an operation B, i.e.: a mixed solution containing 1.5 mmol of DIC, 1.5 mmol of HOBT, and 1.5 mmol of an amino acid with a protecting group is added until a concentration is 0.5 M, the mixture is reacted at 25° C., after 1 hour, the reaction is finished. If the detection by means of the ninhydrin is negative, the reaction is complete. Then the reaction solution is washed 3 times with industrial DMF. Then the operations A and B are performed alternately, and only the corresponding amino acid is added in the operation B along with a synthesis sequence. Until the linking is to Ala, the operation A is performed, finally 1.5 mmol of (Boc)₂₀O and 1.5 mmol of DIPEA in dichloromethane are added, and the mixture is reacted for 30 min to obtain a resin peptide B3 as shown in the following figure.

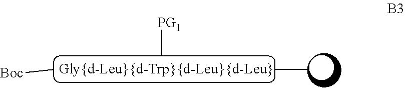

(2) Preparation of a fully protected peptide C3:

a solution of trifluoroisopropanol in dichloromethane with a volume fraction of 33% as a first cleavage reagent is added into the resin peptide B3 obtained in step (1) for cleavage for 3 times with 1 h for each time, and the obtained product is freeze-dried to obtain a white powder, a fully protected peptide C3, with a theoretical molecular weight of 800.75 and an actually detected molecular weight of 800.2.

(3) Preparation of a fully protected peptide thioester D3:

100 mg of the fully protected peptide C3 obtained in step (2) is taken, 1.5 eq of TCFH and 4 eq of NMI in DMF (0.1 M) are respectively added, finally 1.2 eq of p-chlorothiophenol is added, the mixture is reacted at 30° C. for 16 h, and the reaction solution is detected by means of HPLC, a yield is 79%.

(4) Preparation of a peptide thioester E3:

5 mL of a cleavage reagent (a mixed solution of TFA, EDT, phenol, thioanisole

and H₂O at a volume ratio of 87.5 5:2.5:2.5:2.5) is added into the fully protected peptide thioester D3 obtained in step (3), the mixture is reacted for 2 h, the reaction solution is settled with diethyl ether to obtain a crude product peptide thioester, and the peptide thioester is freeze-dried to obtain a powder, a peptide thioester E3, of 82 mg with a yield of 90%.

(5) Preparation of a head-to-tail amide cyclic peptide F3:

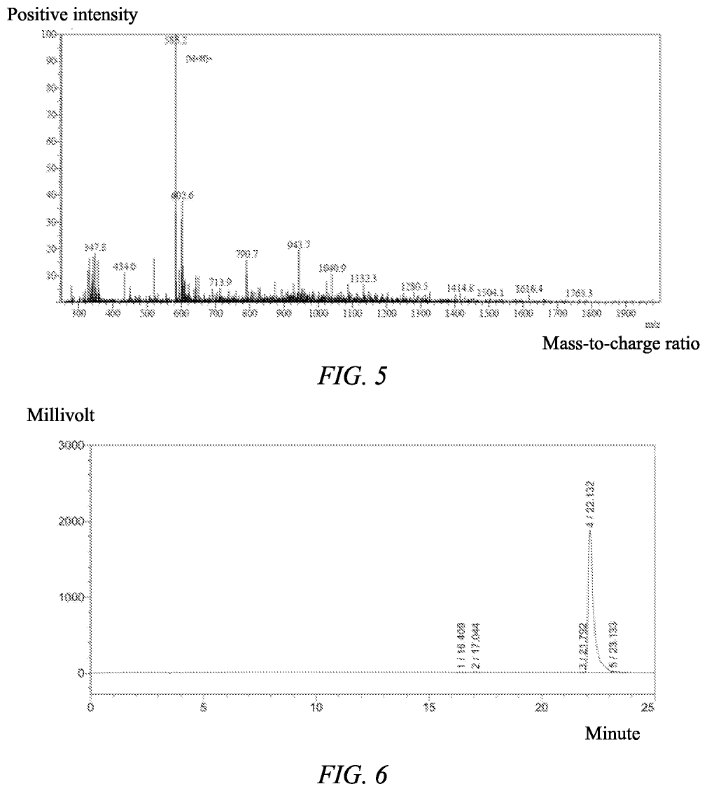

50 mg of the peptide thioester E3 obtained in step (4) is taken, 3.0 eq of DIPEA in DMF (0.0025 M) is added, the mixture is reacted for 12 h; after a solvent is concentrated, the obtained product is settled with diethyl ether to obtain a crude product head-to-tail amide cyclic peptide, and the head-to-tail amide cyclic peptide is dissolved with a water/acetonitrile mixed solution, loaded into a high performance liquid chromatography for separation and purification with a mobile phase of H₂O/0.1 TFA% and ACN/0.1% TFA, and separated and purified with a gradient elution chromatography system via a C18 preparative column. A target fraction is collected. The purity of a collected target peak is detected by an analytical high performance liquid chromatography. A qualified sample is freeze-dried by means of liquid nitrogen and finally the freeze-dried sample is freeze-dried in a vacuum freeze-drying machine to obtain 30 mg of a head-to-tail amide cyclic peptide compound F3 with a yield of 75% and a purity of 98.9%. The head-to-tail amide cyclic peptide compound F3 has a theoretical molecular weight of 582.74 and an actually detected molecular weight of 582.2. A detection is performed at 220 nm, using a C18 (4.6*250 mm) 5 μm chromatographic column, and with a linear gradient from 5% to 65%, acetonitrile (0.05% TFA) in water (0.065% TFA) at speed of 1 mL/min within 25 min, and t_{R}=22.13 min. An MS spectrum and an HPLC profile are as shown in FIG. 5 and FIG. 6.

For the optimization of the reaction conditions in step (3), in example 1, reference can be made to Table 3 and each reference sign in Table 3 has the following meaning: M is a molar feeding ratio of the fully protected peptide C1: the p-chlorothiophenol: the coupling reagent: the alkali. The reaction yield in the table is an HPLC yield of a crude product.

TABLE 3
Preparation of fully protected peptide thioester D1

| | | | | | | Reaction | Reaction | Re- | Re- |
|---|---|---|---|---|---|---|---|---|---|
| Reaction | Re- | Coupling | | | | concen- | tem- | action | action |
| number | actant | agent | Alkali | M | Solvent | tration | perature | time | yield |
| 1 | C1 | HATU | DIPEA | 1:1.2:1.5:4 | DMF | 0.05M | 25° C. | 12 h | 21% |
| 2 | C1 | PyBop | DIPEA | 1:1.2:1.5:4 | DMF | 0.05M | 25° C. | 12 h | 26% |
| 3 | C1 | HBTU | DIPEA | 1:1.2:1.5:4 | DMF | 0.05M | 25° C. | 12 h | 15% |
| 4 | C1 | TBTU | DIPEA | 1:1.2:1.5:4 | DMF | 0.05M | 25° C. | 12 h | 35% |
| 5 | C1 | DIC | — | 1:1.2:1.5:4 | DMF | 0.05M | 25° C. | 12 h | 10% |
| 6 | C1 | TCFH | DIPEA | 1:1.2:1.5:4 | DMF | 0.05M | 25° C. | 12 h | 65% |
| 7 | C1 | TCFH | DIPEA | 1:1.2:1.5:4 | DMSO | 0.05M | 25° C. | 12 h | 55% |
| 8 | C1 | TCFH | DIPEA | 1:1.2:1.5:4 | DMA | 0.05M | 25° C. | 12 h | 50% |
| 9 | C1 | TCFH | NMI | 1:1.2:1.5:4 | DMF | 0.05M | 25° C. | 12 h | 70% |
| 10 | C1 | TCFH | NMI | 1:1.2:1.5:4 | DMF | 0.05M | 25° C. | 4 h | 30% |
| 11 | C1 | TCFH | NMI | 1:1.2:1.5:4 | DMF | 0.05M | 25° C. | 8 h | 40% |
| 12 | C1 | TCFH | NMI | 1:1.2:1.5:4 | DMF | 0.05M | 25° C. | 16 h | 75% |
| 13 | C1 | TCFH | NMI | 1:1.2:1.5:4 | DMF | 0.05M | 25° C. | 24 h | 70% |
| 14 | C1 | TCFH | NMI | 1:1.2:1.5:4 | DMF | 0.05M | 30° C. | 16 h | 78% |
| 15 | C1 | TCFH | NMI | 1:1.2:1.5:4 | DMF | 0.05M | 50° C. | 16 h | 69% |
| 16 | C1 | TCFH | NMI | 1:1.2:1.5:4 | DMF | 0.05M | 100° C. | 16 h | 53% |
| 17 | C1 | TCFH | NMI | 1:1.2:1.5:4 | DMF | 0.1M | 30° C. | 16 h | 82% |
| 18 | C1 | TCFH | NMI | 1:1.2:1.5:4 | DMF | 0.2M | 30° C. | 16 h | 72% |
| 19 | C1 | TCFH | NMI | 1:1.2:1.5:4 | DMF | 0.1M | 30° C. | 16 h | 70% |
| 20 | C1 | TCFH | NMI | 1:2:1.5:4 | DMF | 0.1M | 30° C. | 16 h | 72% |
| 21 | C1 | TCFH | NMI | 1:1.2:1.2:4 | DMF | 0.1M | 30° C. | 16 h | 75% |
| 22 | C1 | TCFH | NMI | 1:1.2:2:4 | DMF | 0.1M | 30° C. | 16 h | 75% |
| 23 | C1 | TCFH | NMI | 1:1.2:1.5:2 | DMF | 0.1M | 30° C. | 16 h | 65% |
| 24 | C1 | TCFH | NMI | 1:1.2:1.5:5 | DMF | 0.1M | 30° C. | 16 h | 72% |

For the optimization of the reaction conditions in step (5), in example 1, reference can be made to Table 4 and each reference sign in Table 4 has the following meaning: M is a molar feeding ratio of the reactants to the alkali. The reaction yield in the table is an HPLC yield of a crude product.

TABLE 4
Preparation of head-to-tail amide cyclic peptide F1

| Reaction | Re- | | | Sol- | Concen- | Reaction | Reaction |
|---|---|---|---|---|---|---|---|
| number | actant | Alkali | M | vent | tration | time | yield |
| 1 | E1 | DIPEA | 1:3 | DMF | 0.005M | 12 h | 75% |
| 2 | E1 | DBU | 1:3 | DMF | 0.005M | 12 h | 62% |
| 3 | E1 | NMI | 1:3 | DMF | 0.005M | 12 h | 70% |
| 4 | E1 | DIPEA | 1:2 | DMF | 0.005M | 12 h | 64% |
| 5 | E1 | DIPEA | 1:5 | DMF | 0.005M | 12 h | 68% |
| 6 | E1 | DIPEA | 1:3 | DMF | 0.0025M | 12 h | 82% |
| 7 | E1 | DIPEA | 1:3 | DMF | 0.01M | 12 h | 40% |

The embodiments of the present invention are not limited to the above-described examples, and various modifications and improvements in forms and details may be made to the present invention by those of ordinary skill in the art without departing from the spirit and scope of the present invention, and the modifications and improvements are considered to fall within the scope of protection of the present invention.

1. A method for synthesizing a peptide thioester, characterized by comprising the following steps:
(1) preparation of a resin peptide B: using a resin A as a carrier and sequentially coupling corresponding amino acids in an order from the C-terminal to the N-terminal according to a target sequence by means of solid-phase synthesis to obtain a resin peptide B;
(2) preparation of a fully protected peptide C: cleaving the resin from the resin peptide B obtained in step (1) with a first cleavage reagent to obtain a fully protected peptide C;
(3) preparation of a fully protected peptide thioester D: subjecting the fully protected peptide C obtained in step (2) to an esterification reaction with p-chlorothiophenol under the action of a coupling agent of TCFH and an alkali in a solvent to obtain a fully protected peptide thioester D; and
(4) preparation of a peptide thioester E: under the action of a second cleavage reagent, removing a protecting group from the fully protected peptide thioester D obtained in step (3) to obtain a peptide thioester E, 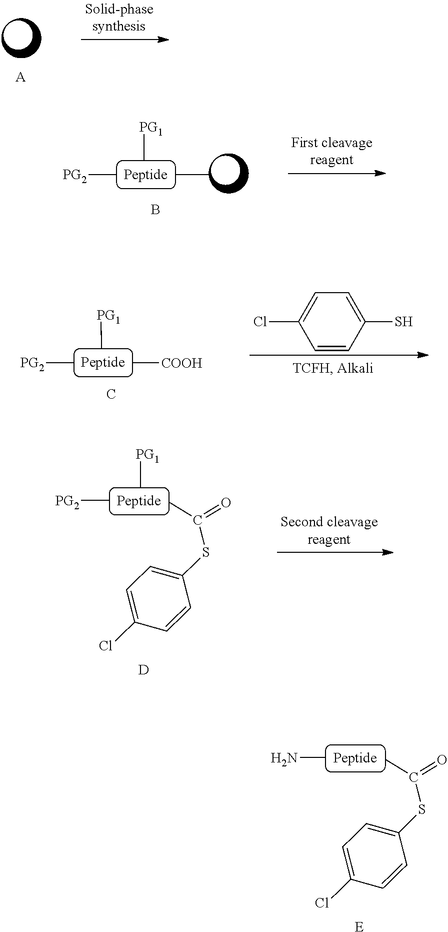
wherein the peptide is a peptide chain and PG₁ is all the protecting groups on a side chain of the peptide chain; and PG₂ is a protecting group at the N-terminal of the peptide chain.

2. The method according to claim 1, characterized in that in step (1), the PG₂ is Boc, the peptide chain is a straight chain, and a procedure for synthesizing the resin peptide B is that the last amino acid of the coupling is a Boc-protected amino acid or an N-terminal Boc protection of the peptide chain is performed by means of di-tert-butyl dicarbonate.

3. The method according to claim 1, characterized in that in step (2), the first cleavage reagent is a dichloromethane solution of trifluoroisopropanol, a volume fraction of the trifluoroisopropanol in the dichloromethane is 10%-90%, and the cleavage is performed for 1-5 times with each cleavage time of 0.5 h-6 h.

4. The method according to claim 3, characterized in that the volume fraction of the trifluoroisopropanol in the dichloromethane is 33%; and the cleavage is performed for 3 times with each cleavage time of 1 h.

5. The method according to claim 1, characterized in that in step (3), the alkali is selected from at least one of DIPEA or NMI; and a molar ratio of the fully protected peptide C, the p-chlorothiophenol, the TCFH, and the alkali is 1:(1-2):(1-3):(2-5).

6. The method according to claim 5, characterized in that the alkali is NMI, and the molar ratio of the fully protected peptide C, the p-chlorothiophenol, the TCFH, and the alkali is 1:1.2:1.5: 4.

7. The method according to claim 1, characterized in that in step (3), the solvent is one or more of DMF, DMSO, or DMA; and a concentration of the fully protected peptide C in the solvent is 0.01 M-0.2 M.

8. The method according to claim 7, characterized in that the solvent is DMF, and the concentration of the fully protected peptide C in the solvent is 0.01 M-0.2 M, preferably, 0.1 M.

9. The method according to claim 1, characterized in that in step (3), the coupling reaction is performed for a time of 4 h-24 h, preferably, 16 h-24 h, more preferably, 16 h.

10. The method according to claim 9, characterized in that the coupling reaction is performed at a temperature of 20° C-100° C., preferably, 25° C-50° C., more preferably, 30° C.

11. The method according to claim 1, characterized in that in step (4), the second cleavage reagent is a mixed solution of one or more of EDT, phenol, thioanisole or H₂O with TFA.

12. The method according to claim 11, characterized in that the second cleavage reagent is a mixed solution of the TFA, the EDT, the phenol, the thioanisole, and the H₂O, and a volume ratio of the TFA, the EDT, the phenol, the thioanisole, and the H₂O in the mixed solution is (50-95):(1-12.5):(1-12.5):(1-12.5):(1-12.5), preferably, 87.5:5:2.5:2.5:2.5.

13. A method for synthesizing a head-to-tail amide cyclic peptide, characterized by comprising the following steps:
(1) preparing a peptide thioester E by a method according to claim 1; and
(2) under the action of an alkali, cyclizing the peptide thioester E prepared in step (1) in a solvent to obtain a head-to-tail amide cyclic peptide F, 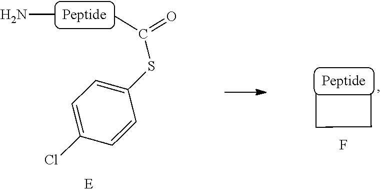
wherein the peptide is a peptide chain.

14. The method according to claim 13, characterized in that in step (2), the alkali is one or more of DIPEA, DBU, imidazole, or NMI, and a molar ratio of the peptide thioester E to the alkali is 1:(2-5).

15. The method according to claim 13, characterized in that in step (2), the solvent is one or more of DMF, DMSO, or DCM; and a concentration of the peptide thioester E in the solvent is 0.001 M-0.01 M.

16. The method according to claim 13, characterized in that in step (2), the alkali is DIPEA; a molar ratio of the peptide thioester E to the alkali is 1:3; and the solvent is DMF, and a concentration of the peptide thioester E in the solvent is 0.0025.
